(19) United States
(12) Patent Application Publication
(10) Pub. No.: US 2022/0204555 A1
(43) Pub. Date: Jun. 30, 2022

(54) METHOD FOR PURIFICATION OF PLASMA PROTEINS

(71) Applicant: CYTIVA BIOPROCESS R&D AB, Uppsala (SE)

(72) Inventors: Martin HALL, Uppsala (SE); Mats ANDER, Uppsala (SE); Mikael BERG, Uppsala (SE); Sandeep KRISTIANSSON, Uppsala (SE)

(21) Appl. No.: 17/607,747

(22) PCT Filed: May 11, 2020
(86) PCT No.: PCT/EP2020/062974

§ 371 Date: Oct. 29, 2021

(30) Foreign Application Priority Data
May 10, 2019 (GB) 1906595.2

(51) Int. Cl. C07K 1/22; C07K 14/755

(52) U.S. Cl. CPC C07K 1/22; C07K 14/755

(57) ABSTRACT

The present invention relates to a method for purification of plasma proteins. More closely, the invention relates to a method using magnetic beads for separation of different plasma proteins from a plasma fraction, such as a cryoprecipitate or cryosupernatant of plasma, or alternatively directly from cell culture of recombinant plasma proteins.

### FIELD OF THE INVENTION

The present invention relates to a method for purification of plasma proteins. More closely, the invention relates to a method using magnetic beads for separation of different plasma proteins from a plasma fraction, such as a cryoprecipitate or cryosupernatant of plasma, or alternatively directly from cell culture of recombinant plasma proteins.

### BACKGROUND

Blood contains different types of cells and molecules which are necessary for vital body functions, and is therefore collected for therapeutic purposes, e.g. for blood transfusions. However, it is possible to separate and prepare different fractions from blood, such as red blood cells or cell-free plasma, which enables a more directed therapeutic treatment of medical conditions. Several proteins in plasma can also be further isolated and used for specific therapeutic treatments, e.g. albumin is used to restore blood volume, immunoglobulins are used for immune deficiencies, and coagulation factors are used for blood coagulation disorders.

Plasma contains proteins of different function, different size, different amount, etc, so there are different methods for purification of the different plasma proteins. The purification processes are often designed to obtain several target proteins from one single starting pool of plasma. The processes typically involve precipitation or chromatography steps or a combination thereof. Chromatography is often used to increase the purity of the target protein and reduce the risk for detrimental side effects. Many plasma proteins exhibit very potent activities, and if present as contaminants, they can cause adverse reactions even at very low levels, when administered to patients.

Collected human plasma is stored frozen, and the initial step in a plasma protein purification process is thawing and pooling of plasma. When thawing at low temperatures, typically 1-6 degrees Celsius, some plasma proteins precipitate and can be collected by e.g. centrifugation. The collected precipitate is called cryoprecipitate, and can be used as a source of e.g. coagulation Factor VIII (FVIII) and von Willebrand Factor (vWF). Most of the FVIII in plasma is present as a complex with the large vWF multimers, and the two proteins are therefore often co-purified. The remaining liquid after removal of the cryprecipitate is often referred to as cryodepleted plasma or cryosupernatant, and this can be used as a source of e.g. albumin, immunoglobulin G (IgG), coagulation Factor IX (FIX).

The purification of many plasma proteins can be challenging. This can depend on the presence of small amounts of contaminants with undesired but potent activity, or that the proteins sometimes lose their activity or gain unwanted activity. For example, the FVIII easily loses activity, and the known methods used for purification are not satisfactory in many respects. Thus, there is a need of improved methods which can be operated at conditions where the proteins retain their activity, in order to obtain plasma products in good yields.

### SUMMARY OF THE INVENTION

The present invention relates to magnetic beads for purification of plasma proteins by batch adsorption of proteins in a crude sample, which has been shown to be a gentle technology that may preserve sensitive proteins. The beads are of chromatography bead type provided with embedded magnetic particles and plasma protein binding ligands.

In a first aspect the invention relates to a method for purification of plasma proteins from a crude sample, comprising binding of desired plasma proteins to ligands on magnetic beads and eluting said plasma proteins, wherein the binding and eluting is performed in batch mode. The method may be performed in large scale to provide large quantities of desired plasma proteins.

The ligands are preferably anion exchange ligands, and are preferably selected from diethylaminoethyl (DEAE), quaternary aminoethyl (QAE) or quaternary ammonium (Q), most preferably the anion exchange ligands are Q-ligands.

Alternatively, the ligands may have affinity to FVIII, such as affinity for the light chain of FVIII.

Preferably the crude sample is a cryoprecipitate and the desired plasma protein(s) are Factor VIII (FVIII) and/or von Willebrand Factor (vWF).

The crude sample may also be a cryosupernatant and the desired plasma protein(s) are albumin, IgG or

Factor IX (FIX). Alternatively, the crude sample may be whole plasma.

In a preferred embodiment, the invention relates to a method according to one or more of the above claims, comprising a) adding a plasma fraction, such as a cryosupernatant or dissolved cryoprecipitate comprising at least one palsma protein to container, such as a bag or tank; b) adding magnetic beads provided with anion ligands, preferably Q ligands, by pouring or pumping said beads into said container;

c) incubating at least 30 minutes with mixing; d) binding plasma proteins to the magnetic beads; e) retaining the magnetic beads with a magnetic field and washing away undesired material, optionally repeated; f) elution of plasma proteins from the magnetic beads in a yield of 92-100% active FVIII from cryoprecipitate or in yield of at least 85% F IX and a FIXa/FIX ratio of less than 1 ‰ from cryosupernatant.

The magnetic beads are preferably porous agarose beads provided with embedded magnetic particles. The invention enables purification in large scale by providing magnetic beads of suitable size and large containers, such as Wave bags, with thereto belonging equipment.

Magnetic chromatography resin prototypes with anion exchange (Q) or affinity (VIII Select) ligands were used for purification of Factor VIII (FVIII), von Willebrand Factor (vWF), or Factor IX (FIX) from human recovered plasma. Conventional packed bed chromatography was performed as a reference for the tests with Q ligand as shown below in the Experimental section.

Although the experiments show purification of plasma-derived proteins, the invention is also contemplated for purification of recombinant plasma proteins directly from cell culture, ie without further purification before binding the plasma proteins to selected ligands on magnetic beads.

### DETAILED DESCRIPTION OF THE INVENTION

The invention provides a magnetic bead capable of binding plasma proteins with a high binding strength and to a high binding capacity. This is achieved with a plasma protein-binding magnetic bead, comprising a porous matrix and one or more magnetic particles embedded in the matrix, where the matrix comprises a porous polymer, preferably agarose, and plasma protein-binding ligands coupled to the porous polymer.

One advantage is that the beads allow the selective binding of large amounts of plasma proteins directly from crude material. A further advantage is that the beads have a favourable adsorption isotherm for plasma proteins, giving a high yield of recovered plasma protein.

The size of the bead may suitably be such that a plurality of beads, to be used in the methods disclosed below, have a volume-weighted median diameter (d50,v) of 8-300 micrometers, such as 20-200, 20-100 micrometers or 20-80 micrometers. Beads of these sizes are easy to retain with a magnetic field, in particular compared to magnetic nanoparticles or micron-sized particles. The mass transport rates are however fast enough to give a rapid uptake of plasma protein by the bead. This applies in particular to beads with median diameter in the 20-100 micrometer and 20-80 micrometer intervals. The bead(s) may be spherical or essentially spherical, e.g. with a sphericity (the surface area of a sphere with the same volume as the bead divided by the surface area of the bead) of at least 0.9.

The invention will now be described in more detail in association with the Experimental part below.

Assays were performed for activity of Factor VIII, Factor IX, Factor IXa (FVIII, FIX, FIXa) or concentration of von Willebrand Factor (vWF).

Comparisons were made between MagSepharose prototypes and non-magnetic resins packed in columns in Experiments 1 and 3 below.

### EXPERIMENTAL PART

Synthesis of Magnetic Bead Prototypes:

MagSepharose Q Prototype, LS-000672

MagSepharose base matrix was washed with 2 M NaOH. Glycidyl trimethylammonium chloride (GMAC) was added and the coupling reaction proceeded at room temperature over night. The GMAC reaction was repeated two more times. The resin was washed with distilled water. The reaction scheme is shown below.

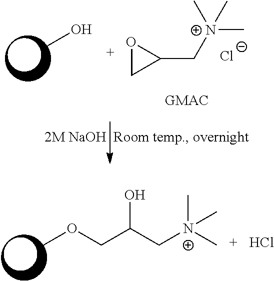

VIII MagSepharose Prototype, LS-034256

NHS-activated MagSepharose was washed with cold 1 mM HCI. VIII ligand was added and the coupling reaction proceeded at pH 8.2 at 24° C. for 2 h 15 min. The resin was washed with 0.1 M acetic acid/0.15 M NaCI pH 4.5, 50 mM Tris/0.15 M NaCI pH 8.5 and distilled water. The reaction scheme is shown below.

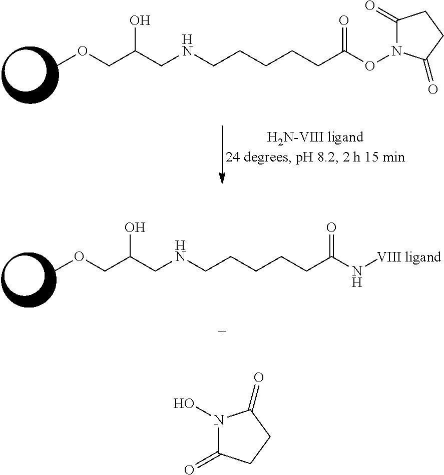

Sample Preparation: Plastic bags with recovered plasma were thawed slowly in ice water (temp approx 0-4° C.), to obtain liquid plasma with cryoprecipitate. The thawed plasma was centrifuged, to collect the cryoprecipitate in the pellet. The supernatant was poured off (cryosupernatant) and the pellets were dissolved in Equilibration buffer (dissolved cryoprecipitate), 1/5 of the original plasma volume.

Analyses: FVIII activity, vWF ELISA (concentration), FIX and FIXa activity: The presence of FVIII, vWF, FIX and FIXa (activated FIX) were analysed using commercial kits according to the manufacturer's instructions. The activities/concentrations are listed as mU/mL (mUnits/mL) in tables below. FVIII activity was determined using the Coamatic FVIII kit from Chromogenix. vWF concentration was determined using the Technozym vWF:Ag ELISA kit from Technoclone. FIX and FIXa activities were determined using commercial kits from Rossix: Rox Factor IX, Rox FIX-A, Factor IXa Calibrator, Factor IXa Control.

Experiment 1: Purification of Factor VIII and von Willebrand Factor From Dissolved Cryoprecipitate Using Q-ligand

A. Packed Chromatography Columns

Chromatography conditions for tests with packed columns:

Columns: HiTrap Q HP 5 mL, HiTrap Capto Q ImpRes 5 mL.

Column volume (CV) 5 mL.

| Method | | Volume | |
|---|---|---|---|
| step | Sample/Buffer | (CV or mL) | |
| Equilibration | 20 mM Na-citrate, 0.15M NaCl, 2.6 | Column | |
| | mM CaCl2, 0.1% Tween 80, pH 7.0 | pre-equilibrated | |
| Sample | Cryoprecipitate dissolved in | 10 | mL |
| | Equilibration buffer | | |
| Wash1 | See Equilibration above | 2 | CV |
| Wash2 | 20 mM Na-citrate, 0.20M NaCl, 2.6 | 7 | CV |
| | mM CaCl2, 0.1% Tween 80, pH 7.0 | | |
| Elution | 0.1M Lysine, 1M NaCl, 10 mM CaCl2, | 5 | CV |
| | 0.1% Tween 80, 12% glycerol, pH 6.0 | | |
| CIP | 0.5M NaOH | 3 | CV |
| Equilibration | See Equilibration above | 10 | CV |

B. Batch Adsorption With Magnetic Beads

Magnetic Beads: MagSepharose Q prototype resin LS-000672, 5 mL resin/tube in tests MagSepharose Q tests were made with 5 mL resin in a 50 mL plastic tube with screw cap. The incubation and mixing of resin and buffer/sample was performed manually by shaking the tube, or in an end-over-end rotating mixer. The tube was then placed in the Sepmag A 200 mL (Sepmag), where the magnetic beads were magnetically attracted to the side of the tube. The clear liquid was removed by plastic Pasteur pipette.

| Method | | Volume |
|---|---|---|
| step | Sample/Buffer | (mL) |
| Equilibration | 20 mM Na-citrate, 0.15M NaCl, 2.6 | Resin pre- |
| | mM CaCl2, 0.1% Tween 80, pH 7.0 | equilibrated |
| Sample | Cryoprecipitate dissolved in | 10 mL |
| | Equilibration buffer | |
| Wash1 | See Equilibration above | 3 × 10 mL |
| | | pooled |
| Wash2 | 20 mM Na-citrate, 0.20M NaCl, 2.6 | 3 × 15 mL |
| | mM CaCl2, 0.1% Tween 80, pH 6.99 | pooled |
| | (spec pH 7.0), cond 23.37 mS/cm | |
| | (spec 21.0 mS/cm?) | |
| Elution | 0.1M Lysine, 1M NaCl, 10 mM CaCl2, | 3 × 10 mL |
| | 0.1% Tween 80, 12% glycerol, pH 6.0 | pooled + |
| | | 10 mL |
| | | separately |
| CIP | 0.5M NaOH | 5 × 20 mL |
| Equilibration | See Equilibration above | 25 mL |
| | | multiple |
| | | times, until |
| | | pH approx 7 |

TABLE 1
Results from Experiment 1
The low limit for quantification (LOQ) was 9 mU/mL for FVIII and
170 mU/mL for vWF. Values below limit of quantification
(LOQ) are indicated by <LOQ.

| | | FVIII | FVIII | FVIII | | vWF | vWF |
|---|---|---|---|---|---|---|---|
| | Volume | act | total | Yield | vWF | total | Yield |
| Fraction | (g = mL) | (mU/mL) | (mU) | (%) | (mU/mL) | (mU) | (%) |
| Q Sepharose HP | | | | | | | |
| Cryoprecipitate | 10.0 | 4276 | 42760 | 100 | 4622 | 46220 | 100 |
| Flow through | 12.2 | <LOQ | | | <LOQ | | |
| Wash1 + 2 | 43.6 | <LOQ | | | 489 | 21320 | 46 |
| Eluate | 4.3 | 8497 | 36537 | 85 | 2717 | 11683 | 25 |
| Capto Q ImpRes | | | | | | | |
| Cryoprecipitate | 10.0 | 4276 | 42760 | 100 | 4622 | 46220 | 100 |
| Flow through | 12.6 | 10 | 126 | 0.3 | <LOQ | | |
| Wash1 + 2 | 43.1 | <LOQ | | | 520 | 22412 | 48 |
| Eluate | 6.1 | 6407 | 39083 | 91 | 515 | 3142 | 6.8 |
| MagSepharose Q | | | | | | | |
| Cryoprecipitate | 10.0 | 4276 | 42760 | 100 | 4622 | 46220 | 100 |
| Flow through | 10.0 | <LOQ | | | <LOQ | | |
| Wash1 | 30.0 | <LOQ | | | <LOQ | | |
| Wash2 | 45.0 | <LOQ | | | 320 | 14400 | 31 |
| Eluate 1-3 | 30.0 | 1454 | 43620 | 102 | 833 | 24990 | 54 |
| Eluate 4 | 10.0 | 114 | 1140 | 2.7 | <LOQ | | |

As shown in Table 1, there were high yields of FVIII in the eluate fractions, and highest yield with the MagSepharose Q prototype resin.

vWF is partially removed during the wash steps without any loss of FVIII.

The results from Experiment 1 surprisingly show that FVIII was obtained in 10-15% higher yields with magnetic beads with Q-ligands than conventional Q-resin.

Experiment 2: Purification of Factor VIII From Dissolved Cryoprecipitate Using VIII Select-ligand

A test was also made with an affinity ligand for FVIII coupled to MagSepharose beads. This magnetic prototype resin was called MagSepharose VIII Select prototype LS-034256. The test was made only with the MagSepharose VIII Select prototype, no comparison was made with a packed column with VIII Select resin. The conditions were comparable to the conditions in Experiment 1, but with different buffers.

TABLE 2
Results from Experiment 2
The low limit for quantification (LOQ) was 9 mU/mL for FVIII and
170 mU/mL for vWF. Values below limit of quantification
(LOQ) are indicated by <LOQ.

| | | FVIII | FVIII | FVIII | | vWF | vWF |
|---|---|---|---|---|---|---|---|
| | Volume | act | total | Yield | vWF | total | Yield |
| Fraction | (g = mL) | (mU/mL) | (mU) | (%) | (mU/mL) | (mU) | (%) |
| MagSepharose VIII | | | | | | | |
| Select | | | | | | | |
| Cryoprecipitate | 10 | 4122* | 41220 | 100 | 4942 | 49420 | 100.0 |
| Eluate 1-3 | 30 | 553 | 16590 | 40.2 | <LOQ | <LOQ | <LOQ |
| Eluate 4 | 10 | 435 | 4350 | 10.6 | <LOQ | <LOQ | <LOQ |

*FVIII activity value from a cryoprecipitate dissolved in equilibration buffer from Experiment 1. Value used for yield estimation in the test with MagSepharose VIII Select prototype.

The FVIII yield was 51% in the eluate fractions (Eluate 1-3 and Eluate 4).

The vWF yield was below LOQ, and a low yield was expected as the affinity ligand binds to FVIII, and vWF which is not in complex with FVIII should not co-purify.

Experiment 3: Purification of Factor IX From Cryosupernatant

A. Packed Chromatography Columns

Chromatography conditions for tests with packed columns:

Columns: HiTrap Q FF 5 mL, HiTrap Capto Q 5 mL. Column volume (CV) 5 mL.

| Method | | Volume | |
|---|---|---|---|
| step | Sample/Buffer | (CV or mL) | |
| Equilibration | 20 mM Na-citrate, 70 mM NaCl, | Column | |
| | pH 7.0 | pre-equilibrated | |
| Sample | Cryosupernatant | 40 | mL |
| Wash | See Equilibration above | 7 | CV |
| Elution | 20 mM Na-citrate, 500 mM NaCl, | 5 | CV |
| | pH 7.0 | | |
| CIP | 0.5M NaOH | 3 | CV |
| PreEquilibration | 20 mM Na-citrate, pH 4.5 | 2 | |
| Equilibration | See Equilibration above | 5 | CV |

A. Batch Adsorption With Magnetic Beads

Magnetic beads: MagSepharose Q prototype resin LS-000672, 5 mL resin/tube in tests

MagSepharose Q tests were made with 5 mL resin in a 50 mL plastic tube with screw cap. The incubation and mixing of resin and buffer/sample was performed manually by shaking the tube, or in an end-over-end rotating mixer. The tube was then placed in a Sepmag A 200 mL (Sepmag) device with adapter for 50 mL tubes, where the magnetic beads were magnetically attracted to the side of the tube. The clear liquid was removed by plastic Pasteur pipette.

| Method | | Volume |
|---|---|---|
| step | Sample/Buffer | (mL) |
| Equilibration | 20 mM Na-citrate, 70 mM NaCl, pH 7.0 | Resin pre- |
| | | equilibrated |
| Sample | Cryosupernatant | 40 mL |
| Wash | See Equilibration above | 3 × 15 mL |
| | | pooled |
| Elution | 20 mM Na-citrate, 500 mM NaCl, pH 7.0 | 3 × 10 mL |
| | | pooled + |
| | | 10 mL |
| | | separately |
| CIP | 0.5M NaOH | 5 × 20 mL |
| Equilibration | See Equilibration above | 25 mL |
| | | multiple |
| | | times, until |
| | | pH approx 7 |

TABLE 3
Results from Experiment 3
The lower limits for quantification (LOQ) were 30 mU/mL for FIX and
0.2 mU/mL for FIXa. Values below limit of quantification
(LOQ) are indicated by <LOQ.

| | | FIX | FIX | FIX | FIXa | FIXa | FIXa | FIXa/ |
|---|---|---|---|---|---|---|---|---|
| | Vol | act | total | Yield | act | total | Yield | FIX |
| Fraction | (g = mL) | (mU/mL) | (mU) | (%) | (mU/mL) | (mU) | (%) | (‰) |
| Q Sepharose FF | | | | | | | | |
| Cryosupernatant | 40.0 | 1365 | 54600 | 100 | 6.2 | 248 | 100 | 4.5 |
| Flow through | 44.1 | <LOQ | | | 0.4 | 18 | 7.1 | 13 |
| Wash | 30.7 | <LOQ | | | <LOQ | | | |
| Eluate | 11.2 | 4148 | 46458 | 85 | 5.7 | 64 | 26 | 1.4 |
| Capto Q | | | | | | | | |
| Cryosupernatant | 40.0 | 1365 | 54600 | 100 | 6.2 | 248 | 100 | 4.5 |
| Flow through | 43.2 | <LOQ | | | 1.4 | 61 | 24 | 47 |
| Wash | 32.2 | <LOQ | | | <LOQ | | | |
| Eluate | 13.8 | 3176 | 43829 | 80 | 2.7 | 37 | 15 | 0.9 |
| MagSeph | | | | | | | | |
| Q tube 1 | | | | | | | | |
| Cryosupernatant | 40.0 | 1365 | 54600 | 100 | 6.2 | 248 | 100 | 4.5 |
| Flow through | 40.0 | <LOQ | | | 0.6 | 24 | 9.7 | 20 |
| Wash | 45.0 | <LOQ | | | <LOQ | | | |
| Eluate 1-3 | 30.0 | 1501 | 45030 | 82 | 0.8 | 24 | 9.7 | 0.5 |
| Eluate 4 | 10.0 | 183 | 1830 | 3.3 | <LOQ | | | |

As shown in the table, FIX was obtained in good yields and the FIXa/FIX ratio was low.

There was a 80-85% yield of FIX activity in the eluate fractions. The FIXa/FIX ratio was lowest in the eluate fraction from the MagSepharose Q prototype resin, indicating low activation of FIX to FIXa.

### CONCLUSION

Batch adsorption with magnetic beads is considered to be a gentle technique, which is an advantage in the purification of sensitive plasma proteins. The present inventors have shown excellent results in yield and activity in the purification of FVIII and vWF in dissolved cryoprecipitate and FIX in cryosupernatant using magnetic beads with suitable ligands. Using large volumes of magnetic beads and instruments for separation enables large-scale applications not possible before.

1. A method for purification of plasma protein(s) from a crude sample, comprising binding of desired plasma protein(s) to ligands on magnetic beads and eluting said plasma protein(s), wherein the binding and eluting is performed in batch mode.

2. The method according to claim 1, wherein the ligands are anion exchange ligands, are preferably selected from diethylaminoethyl (DEAE), quaternary aminoethyl (QAE) or quaternary ammonium (Q), most preferably the anion exchange ligands are Q-ligands.

3. The method according to claim 1, wherein the ligands bind to Factor VIII (FVIII).

4. The method according to claim 3, wherein the ligands have affinity for the light chain of FVIII.

5. The method according to claim 1, wherein the crude sample is a cryoprecipitate and the desired plasma protein(s) are Factor VIII (FVIII) and/or von Willebrand Factor (vWF).

6. The method according to claim 1, wherein the crude sample is a cryosupernatant and the desired plasma protein(s) are albumin, IgG or Factor IX (FIX), preferably FIX.

7. The method according to claim 1, wherein the crude sample is taken directly, without further purification besides settlement of the cells, from cell culture of recombinant plasma proteins.

8. The method according to claim 1, comprising a) adding a plasma protein fraction, such as a cryosupernatant or dissolved cryoprecipitate comprising at least one plasma protein, to a container or bag; b) adding magnetic beads provided with anion ligands by pouring or pumping said beads into said container or bag; c) incubating preferably at least 30 minutes with mixing; d) binding plasma protein(s) to ligands on the magnetic beads; e) retaining the magnetic beads with a magnetic field and washing away undesired material from the magnetic beads, optionally repeated; and f) elution of plasma protein(s) from the ligands on the magnetic beads in a yield of 92-100% active FVIII from cryoprecipitate or in yield of at least 85% F IX and a FIXa/FIX ratio of less than 1 ‰ from cryosupernatant.

9. The method according to claim 1, wherein the magnetic beads are porous agarose beads provided with embedded magnetic particles.

10. The method according to claim 8, wherein the anion ligands are Q ligands.

11. The method according to claim 1, wherein the magnetic beads are 8-300 um in diameter and the method is performed in large scale.
